(19) United States
(12) Patent Application Publication
(10) Pub. No.: US 2014/0017635 A1
(43) Pub. Date: Jan. 16, 2014

(54) Medical treatment apparatus with laser pulses in the femtosecond range

(71) Applicant: Dietmar Fischer, Erlangen (DE)

(72) Inventor: Dietmar Fischer, Erlangen (DE)

(73) Assignee: GME GERMAN MEDICAL ENGINEERING GMBH, Ottensoos (DE)

(21) Appl. No.: 13/548,242

(22) Filed: Jul. 13, 2012

(51) Int. Cl. A61B 18/20; A61C 1/00

(52) U.S. Cl. CPC A61B 18/20; A61C 1/0046
USPC 433/215; 606/9

(57) ABSTRACT

A device for treating skin, the gums, a tooth, or an internal organ by a laser pulse source for generating laser pulses with a pulse duration which is preferably in the femtosecond range, and an optical path for directing the laser pulses from the laser pulse source for the surface of the skin, gums, tooth or internal organ. The laser pulses are directed onto the surface of the skin, gums, tooth or the internal organ in such a way that the pulses are focused in the skin, in the gums, in the tooth or in the internal organ and cause a laser-induced optical breakdown below the surface of the respective skin, gums, tooth or internal organ, whereat the laser pulses have a larger cross-sectional area at the surface of the skin, of the gums, the tooth or of the internal organ than at the location of the laser-induced optical breakdown.

### BACKGROUND OF THE INVENTION

1. Field of the Invention

The present invention relates generally to medical treatment devices that use laser pulses. Particularly, the present invention relates to a skin treatment device, a dental treatment device and a surgical apparatus for treating an internal organ with laser pulses that are focused between the surface of the skin, the gums, a tooth, the internal organ and the location of the laser-induced optical breakdown beneath the surface.

2. Description of the Prior Art

In dermatology, laser systems are, for example, used to remove tattoos, for sclerosing of varicose veins, to remove unwanted pigmentation as well as unwanted hair. The currently used dermatology laser systems have the disadvantage that they damage the skin surface in generating a healing effect in the skin. For example, during a treatment of deeper skin layers, the outer skin layers of the epidermis, as for example the stratum disjunctum, the stratum conjunctum and the stratum lucidum, are damaged, which is undesirable because the healing process is delayed, an open wound is formed with a corresponding risk of infection and the injury of the outer layers of the epidermis is considered as an undesirable side effect by the patient.

### SUMMARY OF THE INVENTION

It is an object of the present invention to provide a medical treatment device with which the skin, the gum, the tooth or an internal organ can be treated with laser radiation at a predetermined depth, without damaging the outer layers of the skin, the gums, the tooth or of the internal organ.

The object of the invention is achieved by a skin treatment device with a laser pulse source for generating laser pulses and an optical path for directing the laser pulses from the laser pulse source to the skin, wherein the laser pulses are applied to the skin so that the pulses are focused into the skin, so that they produce a laser-induced optical breakdown under the skin surface, whereat the laser pulses have a greater cross sectional area on the surface of the skin than at the location of the laser-induced optical breakdown.

Laser-induced optical breakdown is defined, e.g. in Paschotta, R., Encyclopedia of Laser Physics and Technology: Volume A-M, Wiley-VCH, 2008, p. 354. It describes the effect that due to sufficiently high electric field strengths caused by intense laser radiation in an insulating medium, free electrons are accelerated to high energies and start an avalanche process via collisions with other atoms or molecules. The effect of this avalanche process is the creation of a onductive plasma with highly increased absorbance of light. The effect of laser-induced breakdown will be the modification of the material at the location of plasma formation. Depending on the choice of the laser pulse this modification can be, e.g., a change of the refractive index or material removal.

The threshold for laser-induced breakdown can be estimated from experimental data published by Vogel et al. on measurements in water (Vogel, A., et al., Plasma Formation in water by picosecond and nanosecond Nd:YAG laser pulses—Part I: Optical breakdown at threshold and super-threshold irradiance, IEEE Journal of Selected Topics in Quantum Electronics, Vol. 2, No. 4, 1996, p. 847ff) because organic tissue is mainly composed of water. They found threshold light intensities of 4.5×10¹² W/cm² for 30 ps pulses and 0.76×10¹² W/cm² for 6 ns pulses on average.

Results by Stuart (Stuart, B. C., et al., Nanosecond-to-femtosecond laser-induced breakdown in dielectrics, Phys. Rev. B, Vol. 53, No. 4, 1996, p. 1749ff) show that for pulse durations above 20 ps the breakdown threshold is proportional to the square root of the pulse duration whereas for shorter pulse durations the threshold intensity reduces much slower. Experimentally determined threshold values were:
30 ps: 2.3×10¹¹ W/cm²
10 ps: 4.5×10¹¹ W/cm²
1 ps: 2.5×10¹² W/cm²
0.1 ps: 1.7×10¹² W/cm²

According to an embodiment, the laser pulse source generates laser pulses with very short pulse durations in the femtosecond to nanosecond range. In the following the term “femtosecond range” is used for these short pulse durations. The term femtosecond range denotes the pulse duration range from a few femtoseconds up to 100 nanoseconds.

The object of the invention is also achieved by a dental treatment apparatus with a laser pulse source for generating laser pulses and an optical path for directing the laser pulses from the laser pulse source to the gums or a tooth, wherein the laser pulses to the gums or the tooth are focused so that they produce, under the gum surface or the tooth surface, a laser-induced optical breakdown, whereby the laser pulses have a larger cross sectional area on the surface of the gums or the tooth than at the location of the laser-induced optical breakdown. The laser pulse source preferably generates laser pulses with a pulse duration in the femtosecond to nanosecond range. In the following the term “femtosecond range” is used for these short pulse durations. The term femtosecond range denotes the pulse duration range from a few femtoseconds up to 100 nanoseconds.

The object of the invention is also achieved by a surgical device for the treatment of an internal organ with a laser pulse source for generating laser pulses and an optical path for directing the laser pulses from the laser pulse source to the surface of the internal organ, wherein the laser pulses are focused into an inner organ, so that the pulses produce a laser-induced optical breakdown under the surface of the internal organ, whereat the laser pulses have a larger cross sectional area on the surface of the internal organ than at the location of the laser-induced optical breakdown. The laser pulse source preferably generates laser pulses with a pulse duration in the femtosecond to nanosecond range. In the following the term “femtosecond range” is used for these short pulse durations. The term femtosecond range denotes the pulse duration range from a few femtoseconds up to 100 nanoseconds.

The term organ, as used hereinafter includes the skin, mucosa, oral mucosa, the gum, a tooth as well as an internal organ. The internal organs can be for example a colon, prostate, stomach, esophagus, uterus, cervix, lung, pharynx, larynx, brain, heart, and the like. The above-described devices have the advantage that the surface of the organ is not damaged, since the laser pulses have a comparably large cross-sectional area on the surface and thus a low energy density and are focused only on the route to the location of the laser-induced optical breakdown, where the laser pulses have a correspondingly higher energy density. Because of this, damages to the outer tissue layers of the body are avoided, accelerating the healing process and reducing unwanted side effects for patients.

The photo-disruption by the laser-induced optical breakdown may have a diameter of about 0.1 microns to about 50 microns. As the power density of laser pulses at the surface of the organ is lower, the surface of the organ is not damaged. Due to the short laser pulses, a transfer of heat to surrounding tissue is largely avoided. The depth of penetration can be set by the selection of the wavelength of the laser pulses and by the choice of the spot size, i.e., the set cross-sectional area of the organ incident laser pulses on the surface, as well as the focusing angle. The size of the tissue region altered by the laser-induced breakdown may be determined by the peak power and pulse duration. The skin treatment device described above can be used to destroy fat cells in the subcutis, specifically to treat cellulite or the reduced the thickness of fat layers non-invasively. These procedures are also called “body shaping”. The skin treatment device can also be used for skin rejuvenation. By this procedure wrinkles are smoothened and the skin is tightened. The skin treatment device generates small wounds in the skin similar to the so-called fractional skin treatment, which the body then repairs and thereby generates new collagen. In contrast to conventional fractional skin treatments, the proposed treatment device does not cause damage to and/or open wounds in the epidermis.

The laser pulses can be focused at a depth of about 30 microns to about 10 mm below the surface of the organ in order to trigger the laser-induced breakdowns or photo disruptions. For example, the epidermis of the eyelid is only about 30 microns but on the soles about 4 mm thick.

Another application is the selective destruction of unwanted skin lesions located under the top layer of skin. For example, tumor tissue, pigmented lesions, such as age spots, melasma, tattoos and non-pigmented lesions (xanthelasma, nevi, etc.) can be treated.

The surgical device may be used in the field of internal medicine such as urology, gynecology, gastroenterology, etc. With the surgical device, tissue can be removed under the uppermost layer of tissue, for example a tumor or a carcinoma.

By use of the skin treatment device also fat cells in subcutaneous tissues can be destroyed or damaged, whereby the number of fat cells and the fat content of the skin is reduced, which is desirable in fat reduction and cellulite treatment. The uppermost layer of subcutaneous fat cells contains so-called standing fat cell chambers or lobules-like arranged adipose tissue, which are separated from each other by connective tissue. The damage to or destruction of the connective tissue allows fat cells to distribute more evenly in the epidermal layer. With this procedure, the skin treatment device can treat cellulite. The skin treatment device can also treat hair roots and blood vessels. By appropriate choice of treatment depth and the wavelength of the skin treatment device it can also be used for hair removal, vein removal or vascular sclerotherapy. Similarly, acne may be treated by directing the laser pulses on the sebaceous glands in the dermis. Damaged sebaceous glands produce less sebum. A high or excessive sebum production is one of the causes for the outbreak of acne vulgaris or other types of acne. The laser pulses can also be directed on the skin collagen to perform a skin rejuvenation treatment in order to tighten the skin and carry out a wrinkle reduction treatment. The damaging or destroying of collagen results in a wound-healing response in the skin, which means that new collagen is generated and leads to a tighter skin with fewer wrinkles and a smoother skin surface.

There are different types of skin cancer and precursors of skin cancer, so-called precancerous lesions. Depending on the type of cancer or precursor, the cells are in different layers of the skin. With the skin treatment device also cancerous cells can be destroyed by the treatment beam, i.e., the laser pulses are directed to the appropriate treatment areas in the respective depth.

The above-described medical device, i.e., the skin treatment device, the dental treatment device and the surgical device may comprise a focusing device in the optical path, which is arranged so that the laser pulses can be focused during the travel from the surface of the skin, the gums, the tooth or the inner organ to the point of laser-induced breakdown. This can ensure that the laser pulses have a relatively large cross-sectional area and thus a low energy density in the upper tissue layers of the organ so that the upper tissue layers of the organ are not damaged.

The focusing device can be a convex lens which is placed near the surface of the organ. The spot size or cross-sectional area of the pulses on the surface of the organ may vary depending on pulse duration and focusing depth. When focusing is done by the focusing lens, the diameter or cross section of the spot or the cross sectional area on the surface of the organ is between about 50 microns and about 5 cm. The spot or the cross-sectional area of the laser pulse need not be round.

The medical device may be arranged such that the laser pulses have an intensity higher than a self-focusing threshold value of the skin tissue of the gum tissue, the tooth or the tissue of the inner organ on entry into the skin, in the gum, in the tooth or in the organ so that there in the skin, in the gums, in the tooth or in the inner member a self-focusing takes place and leads to the laser-induced optical breakdown under the skin surface, in the gums, in the tooth or in the internal organ.

The self-focusing can be done by the Kerr effect. The Kerr effect is a nonlinear optical effect. When the light intensity exceeds a threshold power, the refractive index changes as a function of the intensity. The Kerr effect is described, for example, in Bergmann Schaefer, Textbook of Experimental Physics, Volume 3, Optics, Edition 10, p. 940 ff, from where the following formulas and values of constants were taken. The Kerr effect can be described by the following formula:

n_{L}=n+δJ;
where n is the refractive index of the medium below the critical power for the non-linearity;
δ is a material-dependent constant;
J is the intensity in W/m²; and
n_{L} is the intensity-dependent refractive index. For water, δ is about 5.4×10⁻²⁰ m² W⁻¹.

The critical power Pₖ, above which the self-focusing occurs, can be estimated by the following formula:

Pₖ=(∈₀c₀λ₀²)/(8πγ_{L});
where Pₖ is the critical power;
∈₀ is the electric field constant;
c₀ is the vacuum speed of light;
λ₀ is the vacuum wavelength; and
γ_{L} is a material constant, which is about 0.5×10⁻²² m² V⁻² for water.

Consequently, the critical power Pₖ, above which self-focusing occurs in water is 1500×10³ W. This value can be used when implementing an embodiment of the invention.

In addition, particularly at short pulse durations more non-linear effects occur. Moreover, the material constants and the critical power can be determined with high effort because the data for δ and γ_{L} vary widely because it is difficult to define experimental setups, especially for short pulse durations (see Bergmann, Schafer, Textbook of Experimental Physics, Volume 3, Optics, 10th edition).

Since the refractive index depends on the intensity above the critical power Pₖ for the self-focusing the penetration depth can be adjusted by the selection of the intensity of the laser pulses and the radius of the light spot on the surface of the skin, of the gums, the tooth or of the internal organ. The higher the intensity of the laser pulses, the more varies the refractive index, the stronger the laser pulses are refracted, and the closer the location of the laser-induced breakdown is to the surface of the organ. The following estimate can be used:

d_{LIOB}=(πnw₀²)/(λ₀(P/P_{K}−1)^{1/2}), P≧P_{K};
where d_{LIOB} is the distance of the laser-induced breakdown to the surface of the organ;
J is the intensity;
n is the refractive index of the medium below the critical power for the non-linearity;
w₀ is the radius of the laser pulse;
λ₀ is the vacuum wavelength;
P_{K} is the critical power, above which self-focusing occurs; and
P is the power of the laser pulse.

The depth of penetration into the tissue also depends on the chosen wavelength. The size of the volume in which the laser-induced breakdown occurs in the tissue can be adjusted by adjusting the pulse duration.

The laser pulses emitted from the laser pulse source have a duration of about 1 fs to about 100 ns, preferably fs from about 10 to about 20 ns, more preferably from about 50 fs to about 10 ns, most preferably from about 50 fs to about 5 ns. Such a laser source is described for example in U.S. Pat. No. 7,131,968 B2.

The spot size or cross-sectional area of the pulses on the surface of the organ may vary depending on pulse duration and focusing depth. When focusing by the Kerr effect, the diameter or cross section of the spot or the cross sectional area on the surface of the organ is between about 50 microns and about 1 mm. The spot or the cross-sectional area of the pulse does not need to be round, as mentioned before.

The laser pulses emitted from the laser pulse source have a wavelength of about 400 nm to about 10,000 nm, preferably from about 700 nm to about 2000 nm, most preferably from about 800 nm to about 1500 nm, most preferably at about 950 nm to about 1400 nm. By selecting the wavelength the penetration depth into the tissue and/or the tissue parts to be treated can be determined. As the laser pulse source for example, a fiber laser, a solid state laser, a Ytterbium-based solid-state laser, a YAG-based solid-state laser, a Cr: Fosterite laser, a Cr: Cunyite laser, a neodymium-doped lithium-yttrium-fluoride laser (YLF be used laser), or a neodymium-doped vanadate laser (YVO4 laser), a semiconductor laser, a slab laser or a diode-pumped solid-state laser. There may be control means to adjust and/or measure one or more of the following parameters of the laser pulse source: pulse repetition rate, pulse duration, energy per pulse, power during the pulse, average power, size of the light spot on the skin surface or the organ surface, depth of the target volume in the skin or in the organ, scan pattern, focusing depth, and/or light wavelength.

The optical path may contain an optical fiber, in particular a hollow optical fiber or a photonic crystal fiber. This allows the laser pulses to be transmitted from the laser pulse source to a movable applicator. The applicator can also be arranged on an endoscope to treat an internal organ minimally invasive.

The optical path may contain at least one movable or immovable mirror and/or at least one lens and/or an exit window. The optical path may be arranged in a pivotable arm with mirrors, a so-called articulating mirror arm. The pivotable arm may contain the aforementioned fiber. The optical path may also have any other type of waveguide for optical radiation.

The optical path may include a scanning device in order to apply the laser pulses on a defined area. The scanning device is particularly suitable for a skin treatment.

The medical device may comprise a measuring device which is adapted to measure or determine the properties of skin, in particular skin type, skin temperature and/or the effects caused by the laser pulses effects in the skin, in the gums, in the tooth or in the internal organ. This allows selection of the appropriate treatments of the organs and/or the monitoring of the treatment of the organ. The laser pulse source can be controlled by the measurement values measured by the measuring device. For example, the measurement values can be used to change the aforementioned parameters for the laser source. This can be performed automatically during the treatment.

The medical device may comprise an imaging device that maps the skin by means of optical coherence tomography or ultrasound. By means of the imaging device, it is possible to monitor the progress of treatment manually or automatically.

The medical treatment apparatus may further comprise a cooling means for cooling the surface of the organ before and/or during and/or after treatment. The patient comfort is increased due to this cooling.

The medical device may further comprise a negative pressure generating means for generating a negative pressure on the surface of the skin, the gums, the tooth or of the internal organ in order to fix the position and/or stretch the surface. By this precise operation conditions can be defined and greater depths of penetration can be achieved. Moreover, the pain for the patient is reduced.

The medical device may comprise a positioning device which is adapted to position the laser pulses in such a way in the skin, in the gums, in the tooth or in the internal organ that the locations of the laser-induced optical breakdowns are adjacent to one another or overlap. This procedure ensures that a tissue, for example, a tumor or cancer is removed completely. The positioning means may be adapted to position the laser pulses in such a way in the skin, in the gums, in the tooth, or in the inner organ that between the locations of the laser-induced optical breakdown not treated tissue does remain. From this healthy tissue the wound healing process of the treated tissue will start. This procedure is especially useful for cosmetic treatments in order to accelerate the post-operative healing because in between the areas treated by laser-induced breakdowns, yet untreated tissue is present. The positioning device may be implemented by the aforementioned scanning device.

The invention also relates to the treatment of the skin, the gums, a tooth or an internal organ with laser pulses. The laser pulses are focused in the skin, in the gums, in the tooth or in the internal organ. The intensity of the laser pulses can be so high that in the skin, in the gums, in the tooth or in the inner organ a self-focusing occurs due to the previously described Kerr effect. The treatment process can be further designed as previously described in connection with the medical device. The laser pulses have preferred pulse duration in the femtosecond range. The term femtosecond range is to be understood that it denotes the pulse duration in the range from a few femtoseconds up to 100 nanoseconds.

The invention will now be illustrated with reference to an exemplary skin treatment device by means of the accompanying drawings.

### BRIEF DESCRIPTION OF THE DRAWINGS

FIG. 1 is a skin treatment device.

FIG. 2 is a skin treatment device with a measuring device and/or an imaging device.

FIG. 3 shows a section through the skin in a case where the focusing is obtained by self-focusing.

FIG. 4 shows a case where the focusing is carried by a focusing lens.

### DETAILED DESCRIPTION OF THE PREFERRED EMBODIMENT

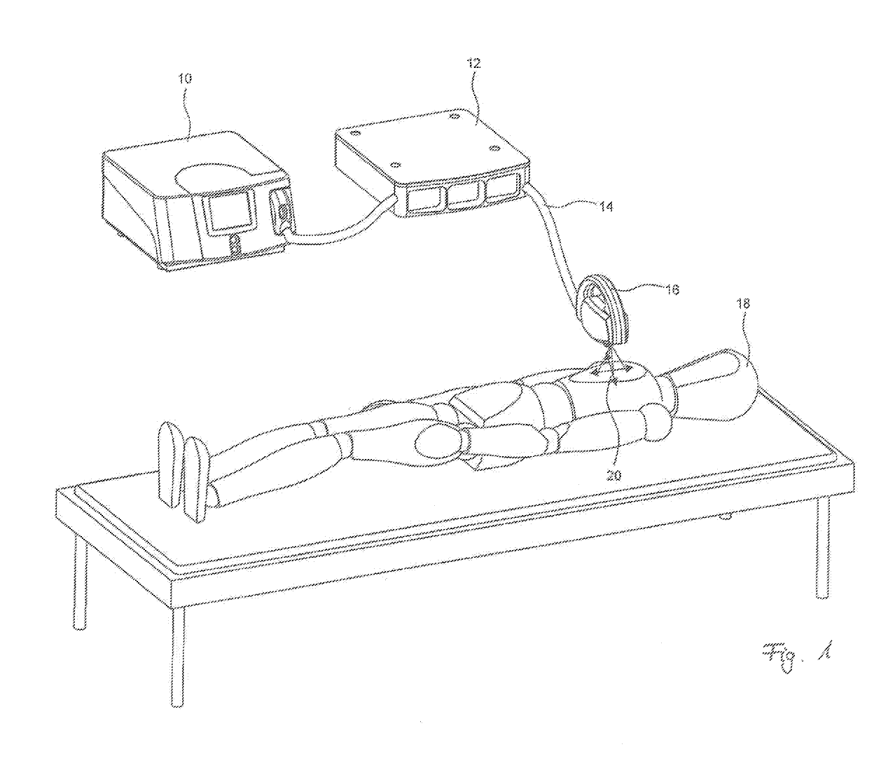

The preferred embodiments of the present invention are illustrated in FIGS. 1-4. FIG. 1 shows a first embodiment of the skin treatment device. A laser pulse source 12 generates laser pulses with a pulse duration which is preferably in the femtosecond range and which are passed through an optical path 14 to an applicator 16. The optical path 14 may comprise an optical fiber, so that the applicator 16 can be moved freely to the desired treatment location 20 on the patient 18. The applicator 16 includes a scanning device (not shown) which applies the laser pulses to a defined treatment area 20. The scanning device may consist of two movable mirrors.

The optical path 14 may contain at least one lens to e.g. adjust the size of the laser pulses. The optical path can be a system of at least one movable or immovable mirror, at least one lens and an optical fiber. In the illustrated embodiment, the optical path 14 is arranged in an articulate mirror arm which can be moved to the treatment site.

It is also possible to form the applicator as a handheld device that the medical staff moves over the treatment area.

The skin treatment device further comprises a control unit 10 into which treatment parameters can be entered via a user interface.

As an example the following treatment parameters may be entered: the pulse repetition rate, pulse duration, the energy per pulse, the power during the pulse, the average power, the size of the light spot on the skin surface, the depth of the region to be treated in the skin, the scan pattern, the focusing depth, the wavelength, the duration of treatment and the dimensions of the treatment area to be covered by the scanning device.

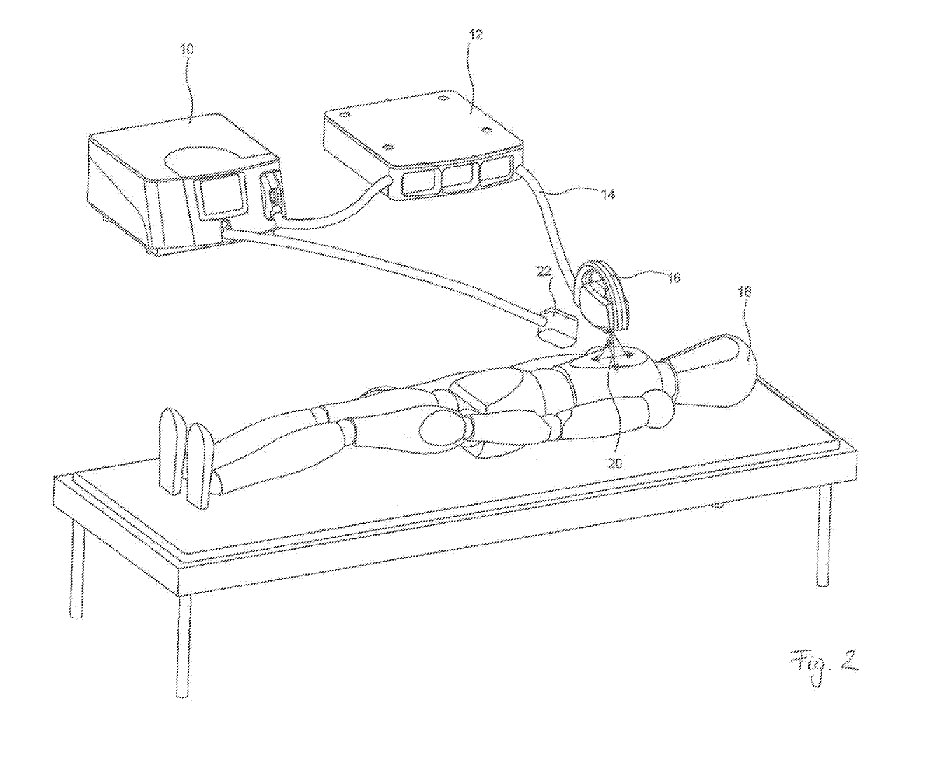

FIG. 2 shows a second embodiment of the skin treatment device. The second embodiment has all the elements of the first embodiment, which will therefore not be described again. The elements are labeled by the same reference numbers as in FIG. 1. In addition to the elements of FIG. 2, the second embodiment contains a sensor device 22. The sensor device 22 may be formed as a measuring device and/or imaging device. The sensor device 22 may include a measuring device which is adapted to measure the properties of the skin, especially the skin type, skin temperature and/or measure/determine the effects caused by the laser pulses in the skin. These values are transmitted to the control device 10, which may propose and/or adjust the treatment parameters. Moreover, it also allows to monitor the progress of treatment. At least one parameter, which is determined from the measured value, may be adapted and/or the treatment discontinued if a patient injury or an unwanted damaged to the tissue could result. The control unit can make the change of the parameters or the termination of the treatment automatically or inform an operator with a reference to a display device.

The sensor device 22 may also include an imaging device that maps the skin by means of optical coherence tomography or ultrasound. Thereby it is possible to make accurate statements about the parameters to be used and to monitor the progress of treatment in more detail. Also these data may be used by the control device 10, as previously described, for the selection of the laser pulse source 12 parameters.

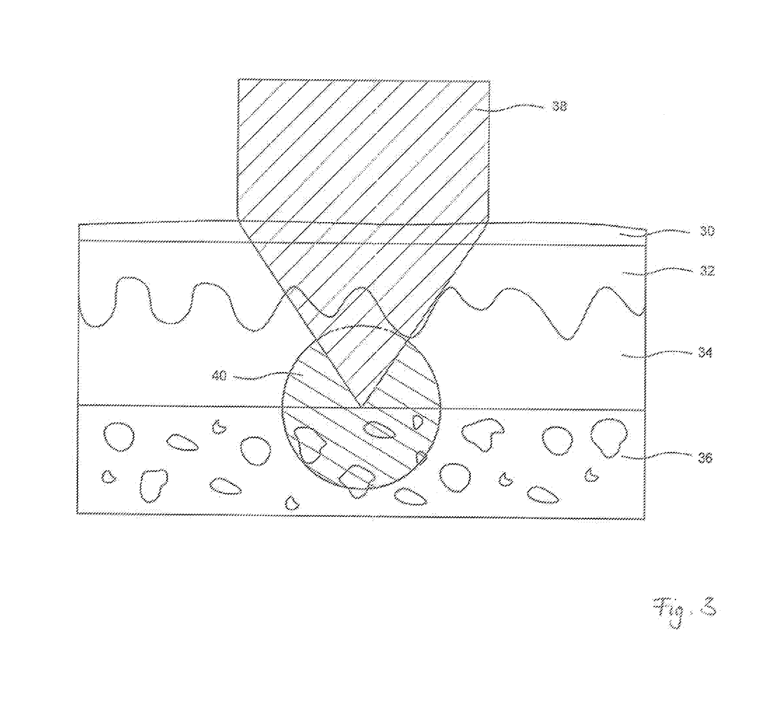

FIG. 3 shows a cross-section of the skin. The skin is described in detail in Roche Lexikon Medizin, 5th Edition, Hoffmann-La Roche AG and Urban & Fischer.

The uppermost skin layer is the stratum corneum 30 as part of the epidermis, which consists of the stratum and the stratum disjunctum conjunctum (not shown). Reference number 32 shows the further layers of the epidermis. Below this is the corium (dermis), which is divided into stratum papilare and stratum reticulare. The lowest layer is called the subcutaneous layer. It contains, e.g., hair roots and subcutaneous fat. The epidermis 30, 32 also contains natural pigments. Unnatural pigments, such as tattoos, are usually located in the dermis 34.

FIG. 3 shows a case in which a substantially parallel light beam 38 in the form of a laser pulse impinges on the stratum corneum 30, i.e., the outer layers of the epidermis. The laser pulse 38 has a higher intensity than the previously described critical power Pₖ, above which the self-focusing occurs due to the Kerr effect. The refractive index of the skin tissue is therefore a function of the intensity J of the laser pulses. Thus there will be a self-focusing of the laser pulse 38.

In FIG. 3 it is shown that the pulse is focused to a point at the transition from the dermis to the subcutis. Thus the laser-induced breakdown takes place on the border between dermis and subcutis. Since the laser pulse has a relatively large spot in the epidermis 30, 32, the energy density of the laser pulse in the epidermis 30, 32 is relatively low. Therefore no or no significant damage is induced on the epidermis 30, 32. In particular, the stratum corneum is not injured, so that the skin is protected from infection as a whole and the side effects for the patient are minimized.

The depth of treatment, i.e. the location of the laser-induced breakdown 40 can be adjusted to the desired treatment such as hair removal, tattoo removal, removal of unwanted pigmentation, fat removal, acne treatment, cellulite treatment, removal of tumors or carcinoma, skin tightening, skin rejuvenation, vascular obliteration, removal of pigmented lesions, etc., or other previously described treatments. In the illustration shown in FIG. 3, hair or fat removal can be performed for example.

The laser pulses can be focused at a depth of about 30 microns to about 10 mm below the surface of the skin in order to trigger the laser-induced breakdown or photo-disruptions there. As an example, the epidermis of the eyelid is only about 30 microns and on the soles of about 4 mm thick.

The spot size or cross-sectional area of the pulses on the surface of the organ may vary depending on pulse duration and focusing depth. When the focusing is done via the Kerr effect, the diameter or cross section of the spot or the cross-sectional area on the surface of the organ is between about 50 microns and about 1 mm. The spot or the cross-sectional area of the pulse need not be round.

The location of the laser-induced breakdown 40 can be adjusted by the intensity of the laser beam 38, since the refractive index is a function of intensity, and by the size of the light spot, since with increasing size of the light spot a longer distance is needed in the skin to focus the laser pulse 38 to an extent that the laser-induced optical breakdown occurs.

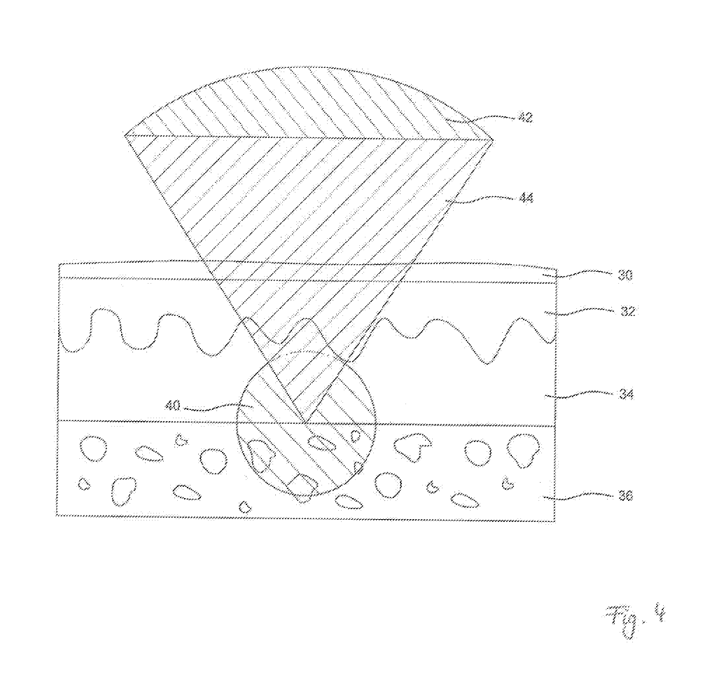

FIG. 4 shows a third embodiment of the invention. FIG. 4 shows a cross-section of the skin, which corresponds to the cross-section in FIG. 3 and thus the description will not be repeated.

The embodiment of FIG. 4 differs from the embodiment according to FIG. 3 that a focusing device is provided in the form of a focusing lens 42, e.g. a convex lens. The laser pulses are focused by the focusing lens to a converging beam 44, which is further focused in the epidermis and dermis. Thereby, the pulses do not need to have such a high intensity as when relying on the Kerr effect alone. Also with this embodiment all the above named treatments can be performed.

The laser pulses can be focused to a depth of about 30 microns to about 10 mm below the surface of the organ in order to trigger the laser-induced breakdowns or photo disruptions. As an example, the epidermis of the eyelid is only about 30 microns and on the soles of about 4 mm thick.

If the focusing is done by the focusing lens, the diameter or cross section of the spot or the cross-sectional area on the surface of the organ is between about 50 microns and about 5 cm. The spot or the cross-sectional area of the pulse need not be round.

The characteristics of the first, second and third embodiments can be combined.

The present invention has the advantage that the tissue can be treated by a photo-disruption in a defined depth of the skin, the gums, a tooth or of an internal organ, which is triggered by a laser-induced optical breakdown. The invention has the further advantage that upper tissue layers of the skin, the gums, the tooth or the internal organ are not damaged, so that the healing process is accelerated and the side effects for the patient are reduced.

During all above-described embodiments, the treated skin can be cooled during one or more of the following time intervals, before the treatment, during the treatment, and after the treatment.

Although the preferred embodiments of the present invention have been described herein, the above description is merely illustrative. Further modification of the invention herein disclosed will occur to those skilled in the respective arts and all such modifications are deemed to be within the scope of the invention as defined by the appended claims.

1. A method for treating of the skin, the gums, a tooth or an internal organ of a patient, the method comprising the steps of:
providing a pulsed laser source for generating a substantially parallel laser beam in the form of laser pulses;
directing the generated laser pulses from the laser source to one of the skin, the gums, a tooth or the internal organ of a patient via an optical path;
generating a laser-induced optical breakdown in the respective skin, gums, tooth or internal organ below the surface of the respective skin, gums, tooth or internal organ, wherein the laser pulses exhibit a larger cross-sectional area on the surface of the respective skin, gums, tooth or internal organ than at the location of the laser-induced optical breakdown and wherein the laser pulses have an intensity which is higher than a self-focusing threshold of the respective skin, gums, tooth or internal organ tissue, so that self-focusing caused by the respective skin, gums, tooth or internal organ occurs on a focus lying in the respective skin, gums, tooth or internal organ tissue below a surface of the respective skin, gums, tooth, or internal organ.

2. The method according to claim 1 wherein the laser pulses have a pulse duration of 1 fs to 100 ns.

3. The method according to claim 1 wherein the laser pulses have a wavelength of about 950 nm to about 1,400 nm.

4. The method according to claim 1 wherein the laser pulses are focused on the beam path from the surface of the skin to the location of the laser-induced optical breakdown.

5. The method according to claim 1 wherein the optical path comprises an optical fiber.

6. The method according to claim 1 further comprising applying the laser pulses over a predetermined area using a scanning device disposed in the optical path.

7. The method according to claim 1 further comprising measuring at least one property of the skin.

8. The method according to claim 7 wherein the measuring at least one property includes measuring a type of skin or measuring a skin temperature.

9. The method according to claim 1, further comprising imaging the skin using optical coherence tomography.

10. The method according to claim 1, further comprising imaging the skin using ultrasound.

11. The method according to claim 1, further comprising cooling the skin before, during, and after the treatment.

12. The method according to claim 1, further comprising generating a negative pressure on the surface of the skin or internal organ.

13. The method according to claim 1, further comprising providing a scanning device for applying and positioning the laser pulses on a plurality of defined treatment areas wherein the laser pulses are positioned in such a way in the skin, in the gums, in the tooth or in the internal organ that the location of the laser-induced optical breakdown in respective ones of the plurality of defined treatment areas are separated from each other by a distance.

14. The method according to claim 1, further comprising providing a scanning device for applying and positioning the laser pulses on a plurality of defined treatment areas wherein the laser pulses are positioned in such a way in the skin, in the gums, in the tooth or in the internal organ that the location of the laser-induced optical breakdown in respective ones of the plurality of defined treatment areas overlap each other.

15. The method according to claim 1, further comprising providing a scanning device for applying and positioning the laser pulses on a plurality of defined treatment areas wherein the laser pulses are positioned in the skin, in the gums, in the tooth or in the internal organ such that tissue not treated by laser pulses remains between the location of laser-induced optical breakdown in respective ones of the plurality of defined treatment areas.
